(19) United States
(12) Patent Application Publication
(10) Pub. No.: US 2009/0171279 A1
(43) Pub. Date: Jul. 2, 2009

(54) Balloon catheter assembly and controller therefor

(75) Inventors: John A. Brumleve, Bloomington, IN (US); Frank J. Fischer, Bloomington, IN (US); Kian Olsen, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, Bloomington, IN (US)

(21) Appl. No.: 12/005,176

(22) Filed: Dec. 26, 2007

(51) Int. Cl. A61M 25/10

(52) U.S. Cl. 604/99.04

(57) ABSTRACT

A balloon catheter assembly included a catheter provided with a distal end and a proximal end and at least one inflation lumen in the catheter. The inflation lumen includes a distal end and a proximal end. An inflatable balloon is provided at the distal end of the catheter and in fluid communication with the distal end of said inflation lumen. The proximal end of the inflation lumen is able to be coupled to a source of fluid. A flow controller is coupled between the inflation lumen and the fluid source, the flow controller being adjustable by an operator of the balloon catheter assembly so as to adjust the flow of inflation fluid to the balloon. In one embodiment, the flow controller includes a manually operable control actuator and a plurality of discrete flow configurations provided in the form of flow through holes in the flow controller. In another embodiment the flow controller includes a manually operable control actuator and a gradually varying flow passage therethrough by means of a coupling channel within the flow controller of varying channel dimension.

### FIELD OF THE INVENTION

The present invention relates to a balloon catheter assembly and to a controller for controlling the rate of inflation of a balloon.

### BACKGROUND OF THE INVENTION

Balloon catheters are widely used in the medical profession for a wide variety of medical applications including angioplasty dilatation, occlusion during medical procedures, urological treatments, venus sampling and pressure monitoring, as well as for deploying implants such as stents and stent grafts.

Typically, a balloon catheter includes a balloon mounted at a distal tip of the catheter assembly and a catheter provided with at least one inflation lumen extending to the interior of the balloon for inflating the balloon. Inflation is effected by an inflation device, well known in the art, located at a proximal end of the assembly, that is outside the patient during the medical procedure. The balloon may also be forcibly deflated, rather than by natural aspiration, through a deflation lumen.

A problem can arise with the speed of inflation and/or deflation of the balloon. Although this can be controlled by the pressure of the supply of inflation or deflation fluid, this is typically set at a compromise pressure for providing adequate inflation and deflation speeds. These set inflation and deflation speeds may not, however, be suitable or appropriate for all deployment procedures.

### SUMMARY OF THE PRESENT INVENTION

The present invention seeks to provide an improved balloon catheter assembly and a flow controller for controlling the rate of inflation of a balloon of a balloon catheter assembly.

According to an aspect of the present invention, there is provided a balloon catheter assembly including a catheter provided with a distal end and a proximal end; at least one inflation lumen in the catheter, said inflation lumen including a distal end and a proximal end; an inflatable balloon at the distal end of the catheter and in fluid communication with the distal end of said inflation lumen; the proximal end of the inflation lumen being able to be coupled to a source of fluid; and a flow controller coupled between the inflation lumen and the fluid source, the flow controller including a variable orifice element for adjusting the flow of inflation fluid to the balloon.

In the preferred embodiment, the flow controller includes a manually operable control actuator.

Advantageously, the catheter includes a deflation lumen. In an embodiment, the inflation lumen and the deflation lumen are the same lumen.

In an embodiment, the variable orifice element of the flow controller includes a plurality of discrete flow configurations. The discrete flow configurations can be provided in the form of flow through holes in the variable orifice element and in use located between the inflation lumen and the source of fluid. In one embodiment, there are provided two flow through holes of different dimensions, to provide first and second flow settings.

In another embodiment, the variable orifice element of the flow controller provides a gradually varying flow passage therethrough. In the preferred embodiment, this is provided by a coupling channel within the flow controller of varying channel dimension.

### DESCRIPTION OF THE DRAWINGS

Embodiments of the present invention are described below, by way of example only, with reference to the accompanying drawings, in which:

FIG. 1 is a side elevational view of a prior art balloon catheter;

FIG. 2 is an enlarged longitudinal cross-sectional view of the balloon of the prior art catheter of FIG. 1;

FIG. 3 is an enlarged transverse cross-sectional view of the catheter shaft of FIG. 1;

FIG. 4 is a schematic diagram in perspective of an embodiment of flow controller;

FIG. 5 is a plan view of an other embodiment of flow controller; and

FIG. 6 is a side elevational view of an actuator of the flow controller of FIG. 5.

### DESCRIPTION OF THE PREFERRED EMBODIMENT

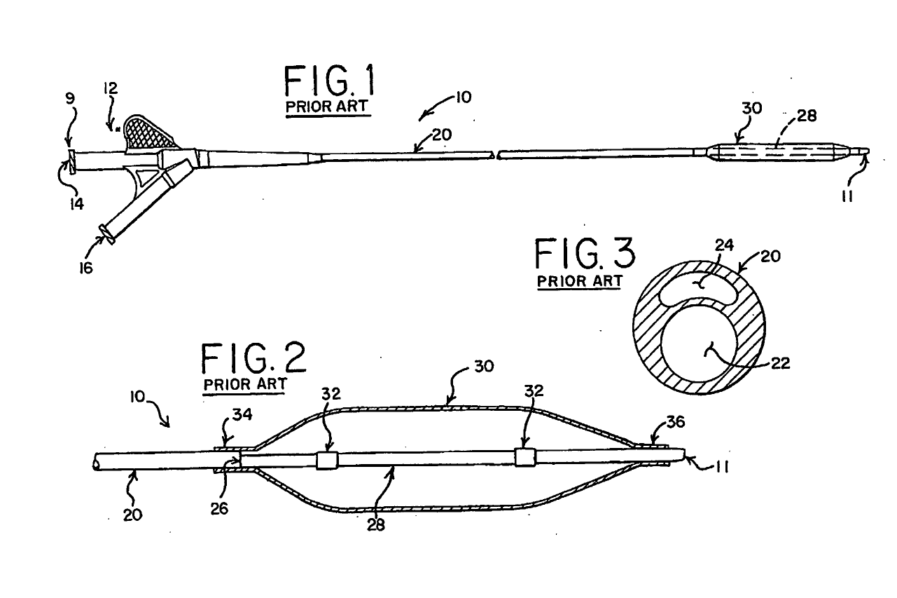

Referring now to the drawings, and particularly to FIGS. 1 to 3, a prior art balloon catheter 10 is shown.

Typically, a balloon catheter 10 has a manifold 12 at the proximal end 9 of the catheter 10 with various ports 14,16. For example, the balloon catheter 10 that is shown has one port 14 for the guide wire 18 and one port 16 for an inflation media as described below. The manifold 12 is attached to a proximal shaft 20 that extends toward the distal end 11 of the catheter 10. As shown in FIG. 3, the proximal shaft 20 may have two different lumens 22, 24 passing longitudinally through the proximal shaft 20. In the example shown, one lumen 22 is for the guide wire 18 and the other lumen 24 is for the inflation media. Thus, the guide wire port 14 of the manifold 12 opens to the guide wire lumen 22, and the inflation port 16 opens to the inflation lumen 24. The described manifold, ports and lumens, however, are only one example of the type of structure that may be used with a balloon catheter and many other examples are possible as well.

At its distal end 26, the proximal shaft 20 may be bonded to an inner shaft 28. As used herein, the term “bonded” simply refers to the boundary between two portions and is not meant to refer to a particular technique for adhering two members together. For example, two shafts may be bonded together by gluing, heat welding, friction welding or the like. However, shafts may also be bonded together by extruding a shaft with two different portions having different shapes, material properties or other characteristics. Furthermore, two members may be attached in various other ways, including with intermediate members disposed therebetween.

As shown in FIG. 2, the inner shaft 28 is smaller in diameter than the proximal shaft 20 and is shifted from the centre axis of the proximal shaft 20 so that the guide wire lumen 22 of the proximal shaft 20 lines up with a matching guide wire lumen 22 extending through the inner shaft 28. Since the inner shaft 28 is smaller in diameter than the proximal shaft 20 and is shifted away from the inflation lumen 24, the inflation lumen 24 is exposed at the distal end 26 of the proximal shaft 20 to the interior of the balloon 30.

In the prior art embodiment shown in FIGS. 1 to 3, the inner shaft 28 extends to the distal end 11 of the catheter 10.

Radio opaque bands 32 may be added to the inner shaft 28 to allow the physician to see the location of the balloon catheter 10 with visualization equipment during intraluminal procedures.

The guide wire lumen 22 of the catheter 10 opens at the distal end 11 of the catheter 10 to allow the catheter 10 to pass over a guide wire 18. The inner shaft 28 is enveloped by balloon 30, which may be used in angioplasty procedures or various other procedures. As shown, the proximal end 34 of the balloon 30 is bonded to both the proximal shaft 20 and the inner shaft 28. However, the proximal end 34 could be bonded to only the proximal shaft 20 or the inner shaft 28 as desired. The distal end 36 of the balloon 30 is bonded to the inner shaft 28. Although various materials may be used for the balloon catheter 10, nylon-based materials, such as polyether block amide (PEBA), which are biocompatible are preferred for most of the components.

Not shown in FIGS. 1 to 3 is the supply of pressurised fluid which is connected to the inflation port 16, typically by a luer-type fitting. Fluid supplies for this purpose are well known in the art so examples are not described herein for the sake of efficiency. The fluid supply of a separate device may also be provided for exhaling the balloon so as to aid in its collapse. Again, devices for this task are well known in the art.

In use, the device of FIG. 1 and 3 is deployed intraluminally into a patient such that the distal end 11 of the catheter 10 is located at the site in the patient to be treated, with the proximal end remaining outside the patient. Once properly located, fluid is fed from the supply through the port 16, into the lumen 24 and hence into the balloon 30 to inflate this. The rate of inflation is typically set by way of the pressure of the fluid supplied and remains substantially constant during the inflation phase, until the fluid supply is closed off by the physician. Deflation of the balloon occurs in a similar fashion.

In some instances, a constant and hard to control rate of inflation or deflation of the balloon can cause treatment difficulties. However, the device of FIGS. 1 to 3 does not permit anything other than relatively crude control of the rate of inflation and deflation of the balloon.

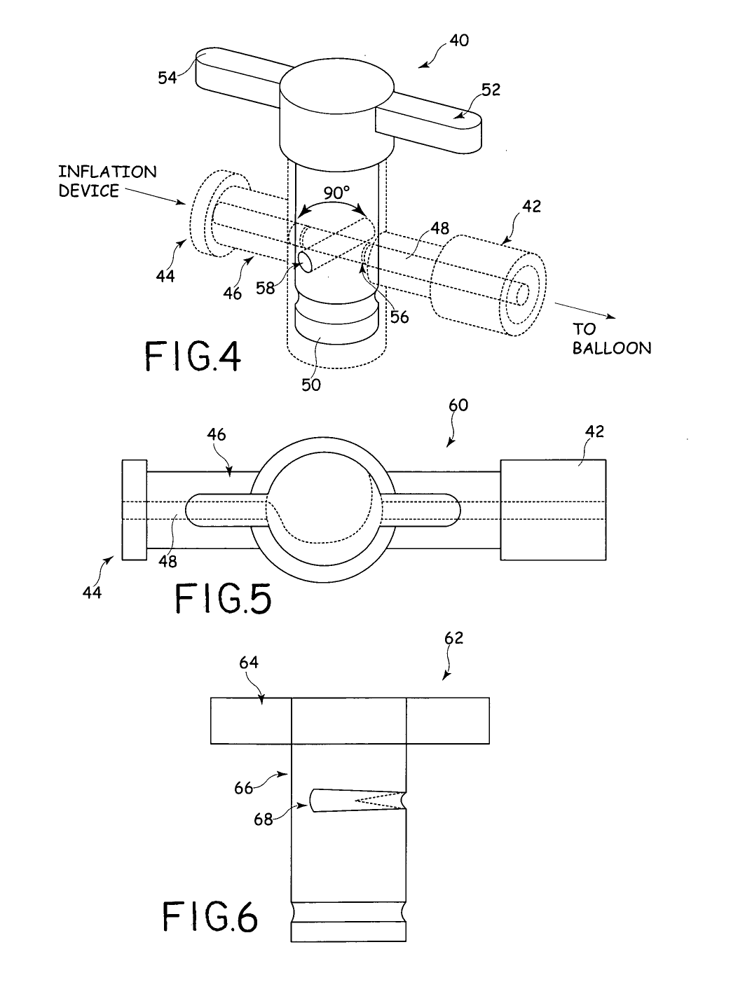

FIG. 4 shows a first embodiment of flow controller 40 which can be coupled to the inflation port 16. For this purpose, the flow controller 40 includes a luer-type lock 42 at one end and a threaded luer-type fitting 44 at the other end, the latter being connectable to the supply of pressurised fluid.

The flow controller 40 includes a body portion 46 with a channel 48 passing therethrough, between the two ends 42 and 44. The body portion 46 is also provided with a chamber 50, of round cross-section, into which a rotatable valve element 52 is located. The valve element 52 includes a handle 54 which allows a physician to turn the valve element during inflation and/or deflation of the balloon.

Within the valve element 52 there are provided two bores 56, 58, the bore 56 having a larger diameter than the bore 58. These are arranged in a crossing fashion as seen in FIG. 4.

When the handle 54 is turned, one of the bores 56, 58 becomes aligned with the channel 48 in the valve body 46, thus fluidly connecting together the two ends 42, 44. When the larger bore 56 is aligned in the channel 48, the flow is relatively high, while when the smaller bore 58 is aligned in the channel 48, the flow is relatively low. Thus, by turning the handle 54, the physician can choose a higher or a lower fluid flow through the inflation lumen 24 and thus a higher or lower inflation and/or deflation rate for the balloon. The valve body 46 can be considered a variable orifice element.

It will also be apparent that flow through the flow controller 40 can be completely stopped by turning the handle by an eighth of a turn or by any amount which causes neither bore 56, 58 to the aligned with the channel 48, in which case the lumen 24 becomes sealed and the balloon remains in the inflated, deflated or partially inflated state until the valve of the flow controller 40 is opened again. This provides a third inflation or deflation condition for the balloon.

Referring now to FIGS. 5 and 6, there is shown a second embodiment of flow controller 60. In this embodiment, the valve element 62 includes a cylindrical stem 66 with a wedge shaped groove 68 extending annularly around the stem 66. As will be apparent particularly form FIG. 5, the groove 68 provides a varying size aperture to the channel 48 as it is rotated and as the groove becomes aligned to different radial extents with the two parts of the channel 48 in the valve body 46. The closer to the wider end of the groove 68 is one end of the channel 48, the wider will be the valve element passage or orifice connecting the two parts of the channel 48. The rotation of the valve element 62 will thus vary the flow through the flow controller. Thus, this embodiment allows the physician to make minute changes in the amount of flow into or out of the inflation lumen 24 and thus make minute changes in the rate of inflation or deflation of the balloon a desired.

What is claimed is:

1. A balloon catheter assembly including a catheter provided with a distal end and a proximal end; at least one inflation lumen in the catheter, said inflation lumen including a distal end and a proximal end; an inflatable balloon at the distal end of the catheter and in fluid communication with the distal end of said inflation lumen; the proximal end of the inflation lumen being able to be coupled to a source of fluid; and a flow controller coupled between the inflation lumen and the fluid source, the flow controller including a variable orifice element for adjusting the flow of inflation fluid to the balloon.

2. A balloon catheter assembly according to claim 1, wherein the flow controller includes a manually operable control actuator.

3. A balloon catheter assembly according to claim 1, wherein the catheter includes a deflation lumen.

4. A balloon catheter assembly according to claim 1, wherein the variable orifice element of the flow controller includes a plurality of discrete flow configurations.

5. A balloon catheter assembly according to claim 4, wherein the discrete flow configurations are provided in the form of flow through holes in the flow controller.

6. A balloon catheter assembly according to claim 5, wherein there are provided two flow through holes of different dimensions.

7. A balloon catheter assembly according to claim 1, wherein the variable orifice element of the flow controller provides a gradually varying flow passage therethrough.

8. A balloon catheter assembly according to claim 7, wherein there is provided a coupling channel within the flow controller of varying channel dimension.

9. A balloon catheter assembly including a catheter provided with a distal end and a proximal end; at least one inflation lumen in the catheter, said inflation lumen including a distal end and a proximal end; an inflatable balloon at the distal end of the catheter and in fluid communication with the distal end of said inflation lumen; the proximal end of the inflation lumen being able to be coupled to a source of fluid; and a flow controller coupled between the inflation lumen and the fluid source, the flow controller being adjustable by an operator of the balloon catheter assembly so as to adjust the flow of inflation fluid to the balloon; wherein the flow controller includes a manually operable control actuator and a plurality of discrete flow configurations provided in the form of flow through holes of different diameters in the flow controller.

10. A balloon catheter assembly including a catheter provided with a distal end and a proximal end; at least one inflation lumen in the catheter, said inflation lumen including a distal end and a proximal end; an inflatable balloon at the distal end of the catheter and in fluid communication with the distal end of said inflation lumen; the proximal end of the inflation lumen being able to be coupled to a source of fluid; and a flow controller coupled between the inflation lumen and the fluid source, the flow controller being adjustable by an operator of the balloon catheter assembly so as to adjust the flow of inflation fluid to the balloon; wherein the flow controller includes a manually operable control actuator and a gradually varying flow passage therethrough by means of a coupling channel within the flow controller of varying channel dimension.
